(19) United States
(12) Patent Application Publication
(10) Pub. No.: US 2007/0026436 A1
(43) Pub. Date: Feb. 1, 2007

(54) GLUCOSE TRANSPORT-RELATED GENES, POLYPEPTIDES, AND METHODS OF USE THEREOF

(75) Inventors: Michael P. Czech, Westborough, MA (US); Abhijit Chakladar, Worcester, MA (US); Adilson L. Guilherme, Shrewsbury, MA (US); Xiaoqing Tang, Lexington, KY (US)

Correspondence Address: FISH & RICHARDSON PC, P.O. BOX 1022, MINNEAPOLIS, MN, 55440-1022, US

(73) Assignee: UNIVERSITY OF MASSACHUSETTS, Boston, MA (US)

(21) Appl. No.: 11/460,102

(22) Filed: Jul. 26, 2006

Related U.S. Application Data
(60) Provisional application No. 60/703,280, filed on Jul. 28, 2005.

(51) Int. Cl. C12Q 1/68; C12Q 1/54; A61K 48/00

(52) U.S. Cl. 435/6; 435/14; 514/44

(57) ABSTRACT

Methods and compositions for modulating glucose transport are provided herein.

### CROSS-REFERENCE TO RELATED APPLICATIONS

This application claims the benefit of priority of U.S. Provisional Patent Application No. 60/703,280, filed Jul. 28, 2005, the contents of which are hereby incorporated by reference in their entirety.

### TECHNICAL HELD

This invention relates to molecular biology, cell biology, glucose transport, and diabetes.

### BACKGROUND

Insulin stimulates glucose transport in muscle and fat. One of the most critical pathways that insulin activates is the rapid uptake of glucose from the circulation in both muscle and adipose tissue. Most of insulin's effect on glucose uptake in these tissues is dependent on the insulin-sensitive glucose sporter, GLUT4 (reviewed in Czech and Corvera, J. Biol. Chem., 274:1865-1868, 1999, Martin et al., Cell Biochem. Biophys., 30:89-113, 1999; Elmendorf et al., Exp. Cell Res., 253:55-62, 1999). The mechanism of insulin action is impaired in diabetes, leading to less glucose transport into muscle and fat. This is thought to be a primary defect in type II diabetes. Potentiating insulin action has a beneficial effect on type II diabetes. This is believed to be the mechanism of action of the drug Rezulin (troglitazone).

Type II diabetes mellitus (non-insulin-dependent diabetes) is a group of disorders, characterized by hyperglycemia that can involve an impaired insulin secretory response to glucose and insulin resistance. One effect observed in type II diabetes is a decreased effectiveness of insulin in stimulating glucose uptake by skeletal muscle. Type II diabetes accounts for about 85-90% of all diabetes cases. In some cases of type II diabetes the underlying physiological defect appears to be multifactoral.

### SUMMARY

The invention is based, at least in par on the discovery of genes and gene products that regulate glucose transport in cells. The genes and gene products described herein are novel targets for modulation for the treatment of disorders in which glucose metabolism is disregulated, such as diabetes.

Accordingly, in one aspect, the invention features methods for identifying a candidate agent that modulates expression or activity of a glucose transport-related polypeptide or nucleic acid encoding the polypeptide. The methods include, for example: (a) providing a sample including a glucose transport-related polypeptide or a nucleic acid encoding the polypeptide, wherein the glucose transport-related polypeptide is a gene product of a gene in Table 1 or Table 2, or a homolog thereof; (b) contacting the sample with a test compound; (c) evaluating expression or activity of the glucose transport-related nucleic acid or polypeptide in the sample; and (d) comparing the expression or activity of the glucose transport-related nucleic acid or polypeptide of (c) to expression or activity of the glucose transport-related nucleic acid or polypeptide in a control sample (e.g., a sample that lacks the test compound, or a reference sample), wherein a change in glucose transport-related nucleic acid polypeptide expression or activity, relative to the control sample, indicates that the test compound is a candidate agent that can modulate the expression or activity of the glucose transport-related nucleic acid or polypeptide.

In various embodiments, the glucose transport-related polypeptide is a gene product of a gene in Table 1, e.g., cyclin-dependent kinase 7 (Cdk7); calcium/calmodulin-dependent protein kinase 1 (Camk1); c-Src tyrosine kinase (Csk); casein kinase 1, alpha 1 (Csnk1a1); casein kinase 1, delta (Csnk1d); casein kinase 1, gamma 2 (Csnk1g); casein kinase 1, gamma 3 (Csnk1g3); casein kinase II, alpha 1 (Csnkca1); discoidin domain receptor family, member 2 Ddr2); eukaryotic elongation factor-2 kinase (Eef1k); epidermal growth factor receptor (EGFR); Ephrin Receptor Eph A7 (Kinase 1, Epha7); endoplasmic reticulum (ER) to nucleus signaling 1 (Ern1); fs/fes-related tyrosine kinase (Fert2); fibroblast growth factor receptor 2 (Fgfr2); Gardner-Rasheed feline sarcoma viral (Fgr) oncogene homolog (Fgr); glycogen synthase kinase 3 beta (Gsk3b); interleukin-1 receptor-associated kinase 1 (Irak1); SH3-binding kinase 1 (SbK); testis-specific kinase 2 (Tesk2); vaccinia related kinase 1 (Vrk1); vaccinia related kinase 2 (Vrk2); or vaccinia related kinase 3 (Vrk3). In various embodiments, the glucose transport-related polypeptide includes an amino acid sequence at least 50, 60, 70, 80, 90, 95, 96, 99, or 100% identical to the gene product of a gene in Table 1. The polypeptide can be a human polypeptide (e.g., a human polypeptide encoded by a gene in Table 1 or a human homolog of a gene in Table 1).

In various embodiments, the glucose transport-related polypeptide is a gene product of a gene in Table 2, e.g., a gene product of one of the following clones from The Institute of Physical and Chemical Research (RIKEN): 9130019I15Rik, 5830417C01Rik, A430091O22Rik, or a human homolog thereof; activin A receptor, type 1 (Acvr1); calcium/calmodulin-dependent serine protein kinase (Cask) or serine/threonine kinase 38 (Stk38). In various embodiments, the glucose transport-related polypeptide includes an amino acid sequence at least 50, 60, 70, 80, 90, 95, 96, 99, or 100% identical to the gene product of a gene in Table 2. In various embodiments, the polypeptide is a human polypeptide.

The samples used in the methods can be or include a cell (e.g., an adipocyte) or can be a cell-free sample. The expression or activity of the glucose transport-related nucleic acid or polypeptide can be evaluated, e.g., using a cell-free or cell-based assay. Modulation of expression can be evaluated using an antibody. In one embodiment, the evaluating includes determining whether glucose transport is modulated in the presence of the test compound, e.g., by determining glucose uptake.

The test compounds evaluated in the methods can be, for example, polynucleotides, polypeptides, small non-nucleic acid organic molecules, small inorganic molecules, or antibodies. For example, the test compounds can be antisense oligonucleotides, inhibitory RNAs, or ribozymes.

Glucose transport may be increased or decreased in the presence of the test compound.

In various embodiments, the glucose transport-related polypeptide is a kinase. In such embodiments, the evaluating can include determining phosphorylation of a substrate by the kinase, e.g., using a kinase assay.

The methods can include steps in which the effect of the test compound on expression or activity of the glucose transport-related polypeptide is evaluated in vivo, e.g., using an animal model, such as an animal model of obesity or diabetes.

In another aspect, the invention features methods for modulating glucose transport in a cell. These methods include, for example; providing a cell; contacting the cell (e.g., in vitro or in vivo) with an agent that modulates expression or activity of a glucose transport-related polypeptide, thereby modulating glucose transport in the cell.

A test compound that modulates expression or activity of a glucose transport-related nucleic acid or polypeptide can be an agent identified by a method described herein, e.g., a method including the following steps: (a) providing a sample including the glucose transport-related polypeptide or a nucleic acid encoding the polypeptide; (b) contacting the sample with a test compound; (c) evaluating expression or activity of the glucose transport-related nucleic acid or polypeptide in the sample; and (d) comparing the expression or activity of the glucose transport-related nucleic acid or polypeptide of (c) to expression or activity of the glucose transport-related nucleic acid or polypeptide in a control sample lacking the test compound, wherein a change in glucose transport-related nucleic acid or polypeptide expression or activity indicates that the test compound is a candidate agent that can modulate the expression or activity of the glucose transport-related nucleic acid or polypeptide.

The test compounds employed in the methods of modulating glucose transport in a cell can modulate the expression or activity of a gene or gene product in Table 1 or Table 2. The test compounds may decrease or increase expression or activity of a gene or gene product in Table 1. The test compounds can be polynucleotides, polypeptides, small non-nucleic acid organic molecules, small inorganic molecules, or antibodies. For example, the test compound can be a small inhibitory RNA. The test compound can also be an antisense oligonucleotide, an inhibitory RNA, or a ribozyme.

The methods for modulating glucose transport in a cell can further include contacting the cell with a second agent that modulates expression or activity of a glucose transport-related polypeptide (or nucleic acid encoding the polypeptide).

The invention also features methods for increasing insulin-stimulated glucose uptake in a subject. The methods include administering to the subject an agent that decreases expression or activity of a gene or gene product in Table 1 in an amount sufficient to modulate glucose metabolism in a cell of the subject, thereby increasing insulin-stimulated glucose uptake in the subject. For example, the subject can be at risk for or suffering from a disorder or condition related to glucose metabolism such as type I diabetes, type II diabetes, or obesity.

The invention also provides methods for modulating glucose metabolism in a subject by administering to the subject an agent that increases expression or activity of a gene or gene product in Table 2 in an amount sufficient to modulate glucose metabolism in a cell of the subject, thereby modulating glucose metabolism in the subject.

Also featured herein are compositions that include a nucleic acid encoding an inhibitory RNA that targets an RNA encoded by a gene in Table 1. In one embodiment, the inhibitory RNA is a small inhibitory RNA.

The invention fierier provides compositions that include an antisense nucleic acid that inhibits the function of a gene product in Table 1.

The invention also features methods for diagnosing a disorder or condition related to glucose metabolism by evaluating the expression or activity of one or more genes or gene products in Tables 1 and 2.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. All cited patents, patent applications, and references (including references to public sequence database entries) are incorporated by reference in their entireties for all purposes. U.S. Provisional App. No. 60/703,280, filed Jul. 28, 2005, is incorporated by reference in its entirety for all purposes.

The details of one or more embodiments of the invention are set forth in the description below. Other features, objects, and advantages of the invention will be apparent from the description and from the claims.

### DETAILED DESCRIPTION

We have identified genes, the polypeptide products of which regulate glucose transport in adipocytes in response to insulin signals. Here, we provide methods and compositions for modulating expression of these polypeptides, and for identifying agents that modulate their expression. The genes encoding the polypeptides we have identified are listed in Tables 1 and 2, below. A first subset of the genes we have identified encodes polypeptides that are negative regulators of glucose transport (Table 1). A second subset encodes positive regulators of glucose transport (Table 2). Decreasing the expression or activity of negative regulators of glucose transport (e.g., via inhibition of gene expression with antisense or siRNA or with agents that inhibit the activity of the gene product) can result in increased glucose uptake, thereby lowering blood glucose levels. Increasing expression or activity of positive regulators (e.g., via overexpression of the gene product or via use of agents that increase the activity of the regulators) can also enhance insulin action, thereby promoting glucose uptake.

Negative Regulators of Glucose Transport

We have found that inhibiting expression of one or more of the genes listed in Table 1 potentiates insulin action by increasing insulin-stimulated glucose uptake. Potentiation of insulin action is beneficial, e.g., in controlling blood glucose action in vivo, e.g., in diabetic patients. Thus, inhibiting the expression or activity of the gene products in Table 1 can be beneficial in the treatment of conditions in which insulin activity or glucose transport is disregulated.

TABLE 1
Negative Regulators of Glucose Transport

| | Murine | Murine | Human | Human |
|---|---|---|---|---|
| | GenBank ® | GenBank ® | GenBank ® | GenBank ® |
| Gene or Gene | Accession No. | Accession No. | Accession No. | Accession No. |
| Product Name | and GI No. | and GI No. | and GI No. | and GI No. |
| (abbreviated) | (nucleotide) | (amino acid) | (nucleotide) | (amino acid) |
| Cdk7 | NM_009874 | NP_034004 | NM_001799 | NP_001790 |
| | GI: 33859521 | GI: 33859522 | GI: 16950659 | NP_001790 |
| Camk1 | NM_133926 | NP_598687 | L41816 | NP_003647 |
| | GI: 19527139 | GI: 19527140 | GI: 790789 | GI: 4502553 |
| Csk | NM_007783 | NP_031809 | NM_004383 | NP_004374 |
| | GI: 31560711 | GI: 31560712 | GI: 4758077 | GI: 4758078 |
| Csnk1a1 | NM_146087 | NP_666199 | NM_001892 | NP_001883 |
| | GI: 22165381 | GI: 22165382 | GI: 34147516 | GI: 19923746 |
| Csnk1d | NM_027874 | NP_082150 | NM_139062 | NP_620693 |
| | GI: 20544146 | GI: 20544147 | GI: 20544144 | GI: 20544145 |
| Csnk1g2 | NM_134002 | NP_598763 | NM_001319 | NP_001310 |
| | GI: 19527223 | GI: 19527224 | GI: 21314777 | GI: 21314778 |
| Csnk1g3 | NM_152809 | NP_690022 | NM_004384 | NP_004375 |
| | GI: 22779896 | GI: 22779897 | GI: 4758079 | GI: 4758080 |
| Csnk2a1 | NM_007788 | NP_031814 | NM_001895 | NP_001886 |
| | GI: 31542426 | GI: 31542427 | GI: 47419902 | GI: 4503095 |
| Ddr2 | NM_022563 | NP_072075 | NM_001014796 | NP_001014796 |
| | GI: 13937362 | GI: 13937363 | GI: 62420885 | GI: 62420886 |
| Eef2k | AK080915 | NP_031934 | NM_013302 | NP_037434 |
| | GI: 26348872 | GI: 6681275 | GI: 25453477 | GI: 9558749 |
| Egfr | NM_207655 | NP_997538 | NM_005228 | NP_005219 |
| | GI: 46560581 | GI: 46560582 | GI: 41327737 | GI: 29725609 |
| Epha7 | NM_010141 | NP_034271 | NM_00440 | NM_004431 |
| | GI: 34328169 | GI: 34328170 | GI: 32967320 | GI: 4758282 |
| Ern1 | NM_023913 | NP_076402 | NM_001433 | NP_001424 |
| | GI: 15284149 | GI: 13249351 | GI: 50346000 | GI: 50346001 |
| Fert2 | U76762 | AAB18988 | NM_005246 | NP_005237 |
| | GI: 1673619 | GI: 1673620 | GI: 4885230 | GI: 4885231 |
| Fgfr2 | NM_010207 | NP_034337 | NM_000141 | NP_000131 |
| | GI: 6753857 | GI: 6758858 | GI: 13186239 | GI: 4503709 |
| Fgr | NM_010208 | NP_034338 | NM_005248 | NP_005239 |
| | GI: 31542815 | GI: 31542816 | GI: 4885234 | GI: 4885235 |
| Gsk3b | NM_019827 | NP_062801 | NM_002093 | NP_002084 |
| | GI: 58000432 | GI: 9790077 | GI: 21361339 | GI: 21361340 |
| Irak1 | NM_008363 | NP_032389 | NM_001569 | NP_001560 |
| | GI: 6680418 | GI: 6680419 | GI: 4755143 | GI: 4504717 |
| SbK | NM_145587 | NP_663562 | NM_001024401 | NP_001019572 |
| | GI: 21704179 | GI: 21704180 | GI: 66773085 | GI: 66773086 |
| Tesk2 | NM_146151 | NP_666263 | NM_007170 | NP_009101 |
| | GI: 31981901 | GI: 31981902 | GI: 6005895 | GI: 6005896 |
| Vrk1 | NM_011705 | NP_035835 | NM_003384 | NP_003375 |
| | GI: 24475937 | GI: 6755985 | GI: 4507902 | GI: 4507903 |
| Vrk2 | NM_027260 | NP_081536 | NM_006296 | NP_006287 |
| | GI: 21312467 | GI: 21312468 | GI: 31543935 | GI: 5454164 |
| Vrk3 | NM_133945 | NP_598706 | NM_016440 | NP_057524 |
| | GI: 19527165 | GI: 19527166 | GI: 31982928 | GI: 31982929 |

Cyclin-Dependent Kinase 7 (Cdk7)

Cyclin-dependent protein kinases such as cdk7 are important regulators of cell cycle progression. Cdk7 forms a multisubunit complex with cyclin H and manage a trois 1 (MAT1). This complex, called CAK (cdk-activating kinase), participates in transcription by phosphorylating the carboxy-terminal domain of the largest subunit of RNA polymerase II and also phosphorylates other CDKs (Fisher and Morgan, Cell, 78(4):713-24, 1994; Shiekhattar et al., Nature, 374: 283-287, 1995). Inhibitors of Cdk7 include the purine derivative, Roscovitine and 5,6-dichloro 1-beta-D-ribofuranosylbenzimidazole (DRB) (Taylor et al., J. Virol., 78(6):2853-62, 2004; te Poele et al., Oncogene, 18(42):5765-72, 1999).

Calcium/Calmodulin-Dependent Protein Kinase I (CamkI)

CamkI is a cytosolic serine/threonine kinase with multiple substrates, such as synapsin I, the cAMP response element binding protein, and the cystic fibrosis conductance regulator (Chin et al., J. Biol. Chem. 272: 31235-31240, 1997). Calcium/calmodulin activates CamkI by binding to the enzyme and by activating CamkI kinase. CamkI activity can be inhibited directly by inhibitors such as KN-93 or by inhibition of calmodulin, e.g., with calmidazolium (Kahl and Means, Oncogene, 18(42):5765-72, 1999).

C-Src Tyrosine Kinase (Csk)

Csk is a cytosolic tyrosine kinase that negatively regulates the Src family kinases by phosphorylation of the Src C-terminal tyrosine (Wang et al., Biochemistry, 40:2004-2010, 2001). Csk modulates Insulin-Like Growth Factor-I (IGF-1) signaling through Src in adipocytes (Sekimoto and Boney, Endocrinology, 144(6):2546-2552, 2003).

Casein Kinase 1 Alpha 1, Delta, and Gamma 2 (Csnk1a1, Csnk1d, Csnk1g2)

Cnsk1 polypeptides are a family of serine/threonine protein kinases highly conserved from yeast to humans. Cnsk1 polypeptides exist in multiple isoforms in mammals. The isoforms localize to discrete cellular components, which is essential for their respective functions (Gross and Anderson, Cell Signal., 10(10):699-711, 1998). Csnk1a1 associates with cytosolic vesicles, the mitotic spindle, and structures within the nucleus (Gross and Anderson, supra). Csnk1a1 binds to retinoid X receptors (RXR) and inhibits apoptosis induced by activated RXR (Zhao et al., J. Biol. Chem., 279(29):30844-30849, 2004). Csnk1a1 is also involved in Wnt/β-catenin signaling according to Liu et al., Cell, 108(6):837-47, 2002.

Cnsk1d binds to microtubules, centrosomes, and the spindle apparatus (Behrend, et al., Eur. J. Cell Biol., 79, 240-251, 2000). Cnsk1d regulates the mammalian circadian clock in conjunction with another Cnsk1 isoform, Cnsk1e (Akashi et al., Mol Cell Biol., 22(6):1693-703, 2002).

Cnsk1g2 phosphorylates and modulates the functions of metastasis-associated protein-1 short form (MTA1s), a polypeptide that sequesters estrogen receptor-alpha in the cytoplasm of breast cancer cells and is thought to contribute to malignancy in breast cancers (Mishra et al., Oncogene, 23(25):4422-9, 2004).

Casein Kinase 2, Alpha 1 (Csnk2a1)

Csnk2 is a tetrameric kinase composed of an alpha, an alpha-prime, and two beta subunits. The alpha subunits contain serine/threonine kinase activity. Upregulation of csnk2 activity is implicated in malignant transformation of human cells (Sarno et al., Pharmacol. Ther., 93(2-3):159-68, 2002). Inhibitors of Csnk2 include apigenin, emodin, DRB, and 4,5,6,7-tetrabromobenzotriazole (TBB) and TBB adducts described in Pagano et al., e.g., 4,5,6,7-tetrabromo-2-(dimethylamino)benzimidazole (2c) (Pagano et al., J. Med. Chem., 47(25):623947, 2004; Song et al., J. Biol. Chem., 275(31):23790-23797, 2000).

Discoidin Domain Receptor Family, Member 2 (Ddr2)

Ddr2 is a receptor tyrosine kinase that interacts with fibrillar collagens (Shrivastava et al., Mol Cell., 1(1):25-34, 1997; Vogel et al., Mol. Cell., (1):13-23, 1997). Collagen binding to Ddr2 leads to Ddr2 autophosphorylation, which in turn triggers downstream signaling events (Leitinger et al., J. Mol. Biol., 344(4):993-1003, 2004).

Elongation Factor-2 Kinase (Eef1k)

Eef2k is a cytosolic calcium/calmodulin serine-threonine kinase. Proteins such as Stress-activated protein kinase 4 and Ribosomal protein S6 kinases inhibit Eef2k activity by phosphorylating specific serines on Eef2k (Knebel et al., EMBO J., 20:4360-4369, 2001; Wang et al., EMBO J., 20:4370-4379, 2001).

Epidermal Growth Factor Receptor (EGFR)

Signals transduced by EGFR, a receptor tyrosine kinase, stimulate cell proliferation, differentiation, and/or survival. EGFR inhibitors include: Erlotinib (Tarceva); Gefitinib (Iressa); PKI166 (Traxler et al., Clin. Cancer Res., 5: 3750s 1999); agents described in Woodburn, Pharmacol. Ther., 82(2-3):241-50, 1999; and EGFR blocking monoclonal antibodies such as those described in Bianco et al., (Curr. Drug Targets, 6(3):275-87, 2005; e.g., ICR62, Cetuximab (Erbitux®), Panitumumab (ABX-EGF), EMD72000 (matuzamab), and IMC-225).

Ephrin Receptor Eph A7 (Epha7; HEK11)

Epha7 is a receptor tyrosine kinase with three different splice variants. Differential expression of Epha7 splice variants regulates cell adhesion during neural fold formation in embryonic development (Holmberg et al., Nature, 408(6809):203-6, 2000).

Endoplasmic Reticulum (ER) to Nucleus Signaling 1 (Ern1; IRE1)

Ern1 is an ER-localized transmembrane protein with intrinsic kinase activity and an endoribonuclease activity. Ern-1 cleaves mRNA of the transcription factor XBP-1 and regulates the unfolded protein response on mammalian cells (Calfon et al., Nature, 420(6912):202, 2002).

Fps/fes-Related Tyrosine Kinase (Fert2; FER)

Fert2 is a non-receptor protein-tyrosine kinase with an amino-terminal Fps/Fes/Fer/CIP4 homology (FCH) domain followed by three regions of predicted coiled-coils (which may regulate oligomerization), a central Src-homology-2 (SH2) domain (which mediates association with phosphotyrosine-containing peptide motifs) and a carboxy-terminal catalytic domain (Greer, Nature Rev. Mol Cell Biol., 3:278-289; 2002).

Fibroblast Growth Factor Receptor 2 (Fgfr2)

Signaling through Fgfrs is mediated primarily by assembly of a multidocking protein complex (Schlessinger, Science, 306(5701):1506-7, 2004). Binding of fibroblast growth factor and heparin sulfate proteoglycan triggers Fgfr dimerization, activation, and autophosphorylation of multiple tyrosine residues in the cytoplasmic domain of the receptor molecule. Proteins phosphorylated in response to fibroblast growth factor stimulation include Shc, Phospholipase-C gamma, STAT1, Gab1 and FRS2 alpha. Phosphorylation of these proteins leads to stimulation of intracellular signaling pathways that control cell proliferation, cell differentiation, cell migration, cell survival, and cell shape (Eswarakumar, Cytokine Growth Factor Rev., 16(2):139-49, 2005).

Gardner-Rasheed Feline Sarcoma Viral Oncogene Homolog (Fgr)

Fgrs are non-receptor tyrosine kinases encoded by members of the c-Src gene family. Fgr inhibits β2-integrin-mediated signaling and Syk kinase function in monocytes. These inhibitory effects are reversed by chemokines and other inflammatory mediators (Vines et al., Immunity, 15(4):507-519, 2001).

Glycogen Synthase Kinase 3 Beta (Gsk3b)

Gsk3b is a proline-directed serine-threonine kinase that was initially identified as phosphorylating and inactivating glycogen synthase. Insulin inhibits Gsk3b by stimulating phosphorylation of an N-terminal serine residue on Gsk3b by Akt (Cross et al., Nature, 378:785-789, 1995). Gsk3b also phosphorylates Eukaryotic protein synthesis initiation factor 2B (eIF2B), thereby inhibiting eIF2B (Welsh et al., Biochem. J., 294:625-629, 1993). Other targets of phosphorylation by Gsk3b include Axin, β-catenin, tau, presenilin-1, cyclin D1, mucin 1, jun, myc, CREB, and others (see, e.g., Frame and Cohen, Biochem. J., 385:1-16, 2001, listing these and other targets of Gsk3b). Inhibitors of Gsk3b include the maleimide compounds, SB-216763 and SB-415286, described in Coghlan et al. (Chem Biol., 7(10):793-803, 2000) and lithium chloride. See also U.S. Pat. Nos. 6,800,632; 6,716,624; 6,608,063; 6,489,344; 6,465,231; 6,417,185; and 6,057,117.

Interleukin-1 Receptor-Associated Kinase 1 (Irak1)

Irak1 is a membrane proximal serine-threonine kinase involved in interleukin-1 (IL-1) signaling that becomes phosphorylated and progressively degraded in response to IL-1 induction. Irak1 associates with IL-1 receptors upon stimulation of the receptors by exposure to IL-1 (Cao et al., Science, 271(5252):1128-1131; 1996). Irak1 has two known functional domains: an N-terminal death domain, which is involved in protein-protein interactions with MyD88 and Toll-interacting protein (Tollip), and a centrally positioned Ser/Thr kinase domain (see Jensen and Whitehead, J. Biol. Chem., 276(31):29037-29044, 2001; and references cited therein).

SH3-Binding Kinase 1 (Sbk1)

Sbk1 includes a serine/threonine protein kinase consensus sequence followed by a C-terminal proline-rich region. In rats, Sbk1 is expressed predominantly in the brain (Nara et al., Eur. J. Biochem., 268:2642-2651, 2001).

Testis-Specific Kinase 2 (Tesk2)

Tesk2 is a serine/threonine kinase expressed predominantly in testicular Sertoli cells. Tesk2 can phosphorylate cofilin and actin-depolymerizing factor (ADF) (Toshima et al., J. Biol. Chem., 276(33):31449-31458, 2001). Phosphorylation of cofilin inactivates cofilin's actin depolymerizing functions and results in the accumulation of actin filaments.

Vaccinia Related Kinases (Vrk1, Vrk2, Vrk3)

The vaccinia related kinases are serine-threonine kinases with sequence homology to the vaccinia virus-encoded B1 kinase (reviewed in Nichols and Traktman, J. Biol. Chem., 279(9):7934-7946, 2004). Vrk1 is highly expressed in proliferating tissues, localizes to nuclei, and regulates phosphorylation of p53. Vrk2 associates with membranes of the endoplasmic reticulum. Vrk1 and Vrk2 both contain COOH-terminal extracatalytic sequences that mediate intracellular localization and phosphorylate casein. Vrk3 lacks kinase activity, but retains domains important for substrate binding.

Positive Regulators of Glucose Transport

Increasing expression or activity of one or more of the genes listed in Table 2 can potentiate insulin action by increasing insulin-stimulated glucose uptake. As discussed for the negative regulators, above, potentiation of insulin action is beneficial, e.g., in controlling blood glucose action in vivo. Thus, increasing the expression or activity of these gene products can be beneficial in the treatment of conditions in which insulin activity or glucose transport is disregulated such as diabetes.

TABLE 2
Positive Regulators of Glucose Transport

| | Murine GenBank ® | Murine | Human | Human |
|---|---|---|---|---|
| Gene or Gene | Accession | GenBank ® | GenBank ® | GenBank ® |
| Product Name | No. | Accession No. | Accession No. | Accession No. |
| (abbreviated) | (nucleotide) | (amino acid) | (nucleotide) | (amino acid) |
| 5830417C01Rik | NM_024282 | NP_077244 | NM_016076 | NP_057160 |
| | GI: 31542015 | GI: 21313498 | GI: 38708308 | GI: 38708309 |
| 9130019I15Rik | BC023755 | AAH23755 | NM_152649 | NP_689862 |
| | GI: 23274128 | GI: 23274129 | GI: 22749322 | GI: 22749323 |
| A430091O22Rik | NM_183024 | NP_898845 | NM_018211 | NP_060681 |
| | GI: 33942105 | GI: 33942106 | GI: 51948365 | GI: 51948366 |
| Acvr1 | NM_007394 | NP_031420 | NM_001105 | NP_001096 |
| | GI: 40254648 | GI: 40254649 | GI: 10862690 | GI: 4501895 |
| Cask | NM_009806 | NP_033936 | NM_003688 | NP_003679 |
| | GI: 6753275 | GI: 6753276 | GI: 4502566 | GI: 4502567 |
| Stk38 | NM_134115 | NP_598876 | NM_007271 | NP_009202 |
| | GI: 19527343 | GI: 19527344 | GI: 31377778 | GI: 6005814 |

Activin A Receptor, Type 1 (Acvr1)

Activins are dimeric growth and differentiation factors which belong to the transforming growth factor-beta (TGF-beta) superfamily of structurally related signaling proteins (Massagué, Annu. Rev. Biochem., 67:753-791, 1998). Activins signal through receptors comprised of activin type I and type II receptors, which are transmembrane serine/threonine kinases. Activin receptors are composed of a ligand-binding extracellular domain with cysteine-rich region, a transmembrane domain, and a cytoplasmic domain with predicted serine threonine specificity.

Calcium/Calmodulin-Dependent Serine Protein Kinase (Cask)

Cask is a member of the membrane-associated guanylate kinase (MAGUK) protein family, which contains multiple domains that mediate protein-protein interactions. Cask contains an N-terminal CaM kinase-like (CKII) domain, an L27 domain, a PDZ domain, an SH3 domain, a Hook domain (also known as the 4.1 binding domain), and C-terminal guanylate kinase-like domain (Lee et al., Mol. Cellular Biol., 22(6):1778-1791, 2002).

Serine/Threonine Kinase 38 (Stk38)

Stk38 (also known as nuclear Dbf2-related (NDR) kinase) is a highly conserved serine/threonine protein kinase regulated by phosphorylation and by the Ca2⁺-binding protein, S100B (Millward et al., EMBO J., 17:5913-5922, 1998). Stk3S8 is efficiently (20-100-fold) activated upon treatment of cells with the protein phosphatase 2A inhibitor, okadaic acid (OA). Stk38 activity is also stimulated by treatment with the Ca2⁺ ionophore A23187 (Tamaskovic et al., J. Biol. Chem., 278(9):6710-6718, 2003).

Screening Assays

Provided herein are methods for identifying modulators of expression or activity of glucose transport-related nucleic acids and polypeptides. The modulators include polypeptides, oligonucleotides, peptidomimetics, carbohydrates, or small molecules such as small organic or inorganic molecules (e.g., non-nucleic acid small organic chemical compounds) that modulate expression (protein or mRNA) or activity of one or more glucose transport-related polypeptides or nucleic acids described herein. In general, assays for identifying the modulators involve determining the effect of a test compound on expression or activity of a glucose transport-related nucleic acid or polypeptide in a test sample (i.e., a sample containing the glucose transport-related nucleic acid or polypeptide). Expression or activity in the presence of the test compound is compared to expression or activity in a control sample (e.g., a sample containing a glucose transport-related polypeptide that was incubated under the same conditions, but without the test compound, or a reference sample). A change in the expression or activity of the glucose transport-related nucleic acid or polypeptide in the test sample compared to the control indicates that the test compound modulates expression or activity of the glucose transport-related nucleic acid or polypeptide.

In various embodiments, assays are provided for screening test compounds that bind to or modulate the activity of a glucose transport-related polypeptide or nucleic acid encoding the polypeptide or biologically active portion thereof. The test compounds to be screened can be obtained using any of the numerous approaches in combinatorial library methods known in the art including: biological libraries; spatially addressable parallel solid phase or solution phase libraries; synthetic library methods requiring deconvolution; the “one-bead one-compound” library method; and synthetic library methods using affinity chromatography selection. The biological library approach is useful for peptide libraries, while the other four approaches are useful for peptide, non-peptide oligomer, or small molecule libraries of compounds (Lam, Anticancer Drug Des., 12:145, 1997).

Examples of methods for the synthesis of molecular libraries can be found in the literature, for example in: DeWitt et al., Proc. Natl. Acad. Sci. USA, 90:6909, 1993; Erb et al., Proc. Natl. Acad. Sci. USA, 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl., 33:2061, 1994; and Gallop et al., J. Med. Chem., 37:1233, 1994.

Libraries of compounds may be presented in solution (e.g., Houghten, Bio/Techniques, 13:412-421, 1992), or on beads (Lam, Nature, 354:82-84, 1991), chips (Fodor, Nature 364:555-556, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA, 89:1865-1869, 1992) or phage (Scott and Smith, Science, 249:386-390, 1990, Devlin, Science, 249:404-406, 1990; Cwirla et al., Proc. Natl. Acad. Sci. USA, 87:6378-6382, 1990; and Felici, J. Mol. Biol., 222:301-310, 1991).

In one embodiment the assay is a cell-based assay in which a cell expressing a glucose transport-related polypeptide (e.g., a gene product of a gene Table 1 or 2), or a biologically active portion thereof, on the cell surface is contacted with a test compound. The ability of the test compound to bind to the polypeptide is then determined. The cell, for example, can be a yeast cell or a cell of mammalian origin. The ability of the test compound to bind to the polypeptide can be determined, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting.

Alternatively, test compounds can be enzymatically labeled with, for example, horseradish peroxidase, alkaline phosphatase, or luciferase, and the enzymatic label detected by determination of conversion of an appropriate substrate to a product. In one embodiment, the assay includes contacting a cell that expresses a membrane-bound form of the glucose transport-related polypeptide, or a biologically active portion thereof, on the cell surface with a known compound that binds to the polypeptide to form an assay mixture, contacting the assay mixture with a test compound, and determining the ability of the test compound to interact with the polypeptide, e.g., by observing whether the test compound preferentially binds to the polypeptide or a biologically active portion thereof as compared to the known compound.

In another embodiment, an assay as described herein is a cell-based assay that includes contacting a cell expressing a membrane-bound form of a glucose transport-related polypeptide, or a biologically active portion thereof, on the cell surface with a test compound, and determining the ability of the test compound to modulate (e.g., stimulate or inhibit) the activity of the polypeptide or biologically active portion thereof. The ability of the test compound to modulate the activity of the polypeptide or a biologically active portion thereof can be determined, for example, by monitoring the ability of the polypeptide to bind to or interact with a target molecule.

The ability of a polypeptide or nucleic acid to bind to or interact with a target molecule can be determined by one of the direct binding methods described herein. As used herein, a “target molecule” is a molecule with which a selected polypeptide or nucleic acid (e.g., a gene or polypeptide encoded by a gene of Table 1 or Table 2, or a homolog thereof) binds or interacts with in nature, for example, a molecule on the surface of a cell that expresses the selected protein, a molecule on the surface of a second cell, a molecule in the extracellular milieu, a molecule associated with the internal surface of a cell membrane, or a cytoplasmic molecule. A target molecule can be a glucose transport-related polypeptide or nucleic acid or some other polypeptide, protein, or nucleic acid. For example, a target molecule can be a component of a signal transduction pathway that facilitates transduction of an extracellular signal (e.g., a signal generated by binding of a compound to a glucose transport-related polypeptide) through the cell membrane and into the cell or a second intercellular protein that has catalytic activity, or a protein that facilitates the association of downstream signaling molecules with a glucose transport-related polypeptide.

The ability of a polypeptide to bind to or interact with a target molecule can also be determined by various other known methods. For example, the activity of the target molecule can be determined by detecting induction of a cellular second messenger of the target (e.g., intracellular Ca²⁺, diacylglycerol, or IP3), detecting catalytic/enzymatic activity of the target on an appropriate substrate (e.g., detecting kinase activity where the glucose transport-related polypeptide is a kinase), detecting the induction of a reporter gene (e.g., a regulatory element that is responsive to a glucose transport-related polypeptide operably linked to a nucleic acid encoding a detectable marker, e.g., luciferase), or detecting a cellular response, for example, glucose uptake, cellular differentiation, or cell proliferation. When the target molecule is a nucleic acid, the compound can be, e.g., a ribozyme or antisense molecule.

In yet another embodiment, an assay as described herein includes contacting a glucose transport-related polypeptide (e.g., a gene product of a gene of Table 1 or 2) or nucleic acid encoding the polypeptide, or biologically active portion thereof, with a test compound and determining the ability of the test compound to bind to the polypeptide or a biologically active portion thereof. Binding of the test compound to the polypeptide can be determined either directly or indirectly as described herein. In one embodiment, the assay includes contacting the polypeptide or biologically active portion thereof with a known compound that specifically binds to the polypeptide to form an assay mixture, contacting the assay mixture with a test compound, and determining the ability of the test compound to interact with the polypeptide (e.g., its ability to compete with binding of the known compound). One can evaluate the ability of the test compound to interact with the polypeptide by determining whether the test compound can preferentially bind to the polypeptide or biologically active portion thereof as compared to the known compound. When the test compound is targeted to a nucleic acid, the binding of the test compound to the nucleic acid can be tested, e.g., by binding, by fragmentation of the nucleic acid (as when the test compound is a ribozyme), or by inhibition of transcription or translation in the presence of the test compound.

In another embodiment, an assay is a cell-free assay that includes contacting a glucose transport-related polypeptide biologically active portion thereof with a test compound and determining the ability of the test compound to modulate (e.g., stimulate or inhibit) the activity of the polypeptide or biologically active portion thereof. For example, determining the ability of the test compound to modulate the activity of the polypeptide can be accomplished by determining the ability of the polypeptide to modify a target molecule. Such methods can, alternatively, measure the catalytic/enzymatic activity of the target molecule on an appropriate substrate.

Many of the gene products in Table 1 and 2 are kinases. For these gene products, one can utilize kinase assays to identify an agent that modulates the activity of the gene product. In general, modulation of an activity of the polypeptide (or a biologically portion thereof), by a kinase assay or another type of assay, is determined by comparing the activity in the absence of the test compound to the activity in the presence of the test compound. For example, to determine the activity of a kinase (e.g., a kinase listed in Table 1 or Table 2) in the presence of a test compound, any standard assay for protein phosphorylation can be carried out. One can use a natural substrate of the kinase or another protein or peptide that the kinase phosphorylates. Assays for kinase activity can also be carried out with biologically active fragments of the kinase (e.g., a fragment that retains catalytic activity).

More specifically, a screen (e.g., a high throughput screen) for kinase inhibitors and agonists can be carried out by: (a) binding one or more types of substrate proteins or peptides to a solid support (e.g., the wells of microtiter plates); (b) exposing the substrate to a blocking agent (standard blocking agents such as bovine serum albumin or gelatin are known); and (c) exposing the substrate to the kinase, a source of phosphate (e.g., ATP with a radioactively labeled gamma-phosphate), and a test compound. The components of the reaction (e.g., the kinase, phosphate source, and test compound) are typically supplied in a buffered solution and the reaction is allowed to proceed at a temperature (the temperature can vary from, for example, room temperature (about 23° C.) to a physiological temperature (about 37° C.)) and for a period of time that is in the linear range of the assay. The reaction can be terminated in a number of ways (by, for example, rinsing the support several times with a buffered solution), and the amount of phosphate incorporated into the bound substrate can be determined (standard techniques are available to measure, for example, radioactive tags). Inhibitors are identified as the agents that reduce the extent to which the kinase was able to phosphorylate the substrate. Agonists are identified as the agents that increase the extent to which the kinase was able to phosphorylate the substrate. See, also, U.S. Pat. No. 6,258,776 for descriptions of other assays that can be used to measure the activity of kinases or a change in the molecules with which a kinase interacts.

In yet another embodiment, the cell-free assay includes contacting a glucose transport-related polypeptide or nucleic acid encoding the polypeptide, or biologically active portion thereof, with a known compound that binds to the polypeptide to form an assay mixture, contacting the assay mixture with a test compound, and determining the ability of the test compound to interact with the polypeptide or nucleic acid by assaying the ability of the polypeptide or nucleic acid to preferentially bind to or modulate the activity of a target molecule (e.g., a target molecule that is a natural substrate or binding partner of the polypeptide).

Cell-free assays are amenable to use of either a soluble form or a membrane-bound form of a polypeptide (if the polypeptide is a membrane-containing polypeptide. In the case of cell-free assays including a membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100®, Triton X-114®, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPS), and N-dodecyl-N,N-dimethyl-3-ammonio-1-propane sulfonate.

In certain embodiments of the new assay methods, it may be desirable to immobilize either the glucose transport-related polypeptide or its target molecule to facilitate separation of complexed from uncomplexed forms of one or both of the proteins, as well as to automate the assay. Binding of a test compound to the polypeptide, or interaction of the polypeptide with a target molecule in the presence and absence of a test agent, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or a glucose transport-related polypeptide, and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or activity of the polypeptide can be determined using standard techniques.

Other techniques for immobilizing proteins on matrices can also be used in the screening assays. For example, either the glucose transport-related polypeptide or its target molecule can be immobilized utilizing conjugation of biotin and streptavidin. Biotinylated polypeptides or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide or target molecules, but which do not interfere with binding of the glucose transport-related polypeptide to its target molecule, can be derivatized to the wells of the plate, and unbound target or polypeptide trapped in the wells by antibody conjugation. Methods for detecting such complexes such as GST-immobilized complexes, include immunodetection of complexes using antibodies reactive with the polypeptide or target molecule, as well as enzyme-linked assays which rely on detecting an enzymatic activity associated with the polypeptide or target molecule.

In another embodiment, modulators of expression of a polypeptide are identified in a method in which a cell is contacted with a test compound and the expression of the selected mRNA or protein (e.g., the mRNA or protein corresponding to a glucose transport-related polypeptide or gene encoding the polypeptide, e.g., in Table 1 or 2) in the cell is determined. The level of expression of the selected mRNA or protein in the presence of the test compound is compared to the level of expression of the selected mRNA or protein in the absence of the test compound. The test compound can then be identified as a modulator of expression of the polypeptide (i.e., a candidate compound) based on this comparison. For example, when expression of the selected mRNA or protein is greater (statistically significantly greater) in the presence of the test compound than in its absence, the test compound is identified as a candidate agent that is a stimulator of the selected mRNA or protein expression. Alternatively, when expression of the selected mRNA or protein is less (statistically significantly less) in the presence of the test compound than in its absence, the test compound is identified as a candidate agent that is an inhibitor of the selected mRNA or protein expression. The level of the selected mRNA or protein expression in the cells can be determined by methods described herein.

In yet another aspect, a glucose transport-related polypeptide can be used as a “bait protein” in a two-hybrid assay or three hybrid assay (see, e.g., U.S. Pat. No. 5,283,317; Zervos et al., Cell, 72:223-232, 1993; Madura et al., J. Biol. Chem. 268:12046-12054, 1993; Bartel et al., Bio/Techniques 14:920-924, 1993; Iwabuchi et al., Oncogene 8:1693-1696, 1993; and PCT Publication No. WO 94/10300), to identify other proteins that bind to or interact with the glucose transport-related polypeptide and modulate activity of the polypeptide. Such binding proteins are also likely to be involved in the propagation of signals by the glucose transport-related polypeptide as, for example, downstream elements of the signaling pathway involving glucose transport.

Antisense Nucleic Acids

Agents to modulate the expression of the glucose transport-related polypeptides described herein include antisense nucleic acid molecules, i.e., nucleic acid molecules whose nucleotide sequence is complementary to all or part of an mRNA based on the sequence of a gene encoding glucose transport-related polypeptide (e.g., based on a sequence of a gene listed in Table 1 or Table 2). An antisense nucleic acid molecule can be antisense to all or part of a non-coding region of the coding strand of a nucleotide sequence encoding the glucose transport-related polypeptide. Non-coding regions (“5′ and 3 untranslated regions”) are the 5′ and 3′ sequences that flank the coding region in a gene and are not translated into amino acids.

Based upon the sequences disclosed herein, one of skill in the art can easily choose and synthesize any of a number of appropriate antisense molecules to target a gene described herein. For example, a “gene walk” comprising a series of oligonucleotides of 15-30 nucleotides spanning the length of a nucleic acid described in Table 1 can be prepared, followed by testing for inhibition of expression of the gene. Optionally, gaps of 5-10 nucleotides can be left between the oligonucleotides to reduce the number of oligonucleotides synthesized and tested.

An antisense oligonucleotide can be, for example, about 5, 10, 15, 20, 25, 30, 35, 40, 45, or 50 nucleotides or more in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules described herein can be prepared in vitro and administered to an animal, e.g., a mammal, e.g., a human patient. Alternatively, they can be generated in situ such that they hybridize with or bind to cellular mRNA and/or genomic DNA encoding a selected polypeptide described herein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. For example, to achieve sufficient intracellular concentrations of the antisense molecules, vector constructs can be used in which the antisense nucleic acid molecule is placed under the control of a strong pol II or pol III promoter.

An antisense nucleic acid molecule used to modulate expression or activity of a glucose transport-related polypeptide can be an α-anomeric nucleic acid molecule. An α-anomeric nucleic acid molecule forms specific double-stranded hybrids with complementary RNA in which, contrary to the usual, β-units, the stands run parallel to each other (Gaultier et al., Nucleic Acids Res. 15:6625-6641, 1987). The antisense nucleic acid molecule can also comprise a 2′-o-methylribonucleotide (Inoue et al., Nucleic Acids Res., 15:6131-6148, 1987) or a chimeric RNA-DNA analog (Inoue et al., FEBS Lett., 215:327-330, 1987).

Antisense molecules that are complementary to all or part of a glucose transport-related gene are also useful for assaying expression of such genes using hybridization methods known in the art. For example, the antisense molecule is labeled (e.g., with a radioactive molecule) and an excess amount of the labeled antisense molecule is hybridized to an RNA sample. Unhybridized labeled antisense molecule is removed (e.g., by washing) and the amount of hybridized antisense molecule measured. The amount of hybridized molecule is measured and used to calculate the amount of expression of the glucose transport-related gene. In general, antisense molecules used for his purpose can hybridize to a sequence from a glucose transport-related gene under high stringency conditions such as those described herein. When the RNA sample is first used to synthesize cDNA, a sense molecule can be used. It is also possible to use a double stranded molecule in such assays as long as the double-stranded molecule is adequately denatured prior to hybridization.

Ribozymes

Also provided are ribozymes that have specificity for sequences encoding the glucose transport-related polypeptides described herein (e.g., for a sequence of a gene in Table 1 or Table 2). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature, 334:585-591, 1988)) can be used to catalytically cleave mRNA transcripts to thereby inhibit translation of the protein encoded by the mRNA. A ribozyme having specificity for a nucleic acid molecule of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a glucose transport-related mRNA (Cech et al. U.S. Pat. No. 4,987,071; and Cech et al., U.S. Pat. No. 5,116,742). Alternatively, an mRNA encoding a glucose transport-related polypeptide can be used to select a catalytic RNA having a specific ribonuclease activity from a pool of RNA molecules. See, e.g., Bartel and Szostak, Science, 261:1411-1418, 1993.

Also provided herein are nucleic acid molecules that form triple helical structures. For example, expression of a glucose transport-related polypeptide can be inhibited by targeting nucleotide sequences complementary to the regulatory region of the gene encoding the polypeptide (e.g., the promoter and/or enhancer) to form triple helical structures that prevent transcription of the gene in target cells. See generally Helene, Anticancer Drug Des. 6(6):569-84, 1991; Helene, Ann. N.Y. Acad. Sci., 660:27-36, 1992; and Maher, Bioassays, 14(12):807-15, 1992.

In various embodiments, nucleic acid molecules (e.g., nucleic acid molecules used to modulate expression of a glucose transport-related polypeptide) can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., Bioorganic & Medicinal Chem., 4(1): 5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., Proc. Natl. Acad. Sci. USA, 93: 14670-675, 1996.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup, 1996, supra; or as probes or primers for DNA sequence and hybridization (Hyrup, 1996, supra; Perry-O'Keefe et al., Proc. Natl. Acad. Sci. USA, 93: 14670-675, 1996).

PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup, 1996, supra, and Finn et al., Nucleic Acids Res., 24:3357-63, 1996. For example, a DNA chain can be synthesized on a solid support using standard phosphoramidite coupling chemistry and modified nucleoside analogs. Compounds such as 5′-(4 methoxytrityl)amino-5′-deoxy-thymidine phosphoramidite can be used as a link between the PNA and the 5′ end of DNA (Mag et al., Nucleic Acids Res., 17:5973-88, 1989). PNA monomers are then coupled in a stepwise manner to produce a chimeric molecule with a 5′ PNA segment and a 3′ DNA segment (Finn et al., Nucleic Acids Res., 24:3357-63, 1996). Alternatively, chimeric molecules can be synthesized with a 5′ DNA segment and a 3′ PNA segment (Peterser et al., Bioorganic Med. Chem. Lett., 5:1119-11124, 1975).

In some embodiments, the oligonucleotide includes other appended groups such as peptides (e.g., for targeting host cell receptors in vivo), or agents facilitating transport across the cell membrane (see, e.g., Letsinger et al., Proc. Natl. Acad. Sci. USA, 86:6553-6556, 1989; Lemaitre et al., Proc. Natl. Acad. Sci. USA, 84:648-652, 1989; WO 88/09810) or the blood-brain barrier (see, e.g., WO 89/10134). In addition, oligonucleotides can be modified with hybridization-triggered cleavage agents (see, e.g., Krol et al., Bio/Techniques, 6:958-976, 1988) or intercalating agents (see, e.g., Zon, Pharm. Res., 5:539-549, 1988). To this end, the oligonucleotide can be conjugated to another molecule, e.g., a peptide, hybridization triggered cross-linking agent transport agent, or hybridization-triggered cleavage agent.

RNA Interference

Another means by which expression of glucose transport-related polypeptides can be modulated is by RNA interference (RNAi). RNAi is a process in which mRNA is specifically degraded in host cells, thereby silencing expression of the gene encoding the mRNA. RNA-mediated gene silencing can be induced in mammalian cells in many ways, e.g., by enforcing endogenous expression of RNA hairpins (see Paddison et al., Proc. Natl. Acad. Sci. USA, 99:1443-1448, 2002) or, by transfection of small (21-23 nt) double stranded (dsRNA) (e.g., by cationic liposome transfection and electroporation), by introduction of a dsRNA to a cavity or intercellular space or by introduction of DNA encoding the dsRNA. See, e.g., Caplen, Trends in Biotech., 20:49-51, 2002. Methods for modulating gene expression with RNAi are described, e.g., in U.S. Pat. No. 6,506,559 and U.S. Pat. Pub. No. 20030056235.

In various embodiments, dsRNA corresponding to a portion of the gene to be silenced (e.g., a gene encoding a glucose sport-related polypeptide, e.g., a gene of Table 1; a gene encoding a polypeptide that activates expression or activity of a gene product listed in Table 1; or a gene encoding a polypeptide that inhibits a gene product listed in Table 2), or DNA encoding the dsRNA, is introduced into a cell. The dsRNA is digested into 21-23 nucleotide-long duplexes called short interfering RNAs (or siRNAs), which bind to a nuclease complex to form what is known as the RNA-induced silencing complex (or RISC). The RISC targets the homologous transcript by base pairing interactions between one of the siRNA strands and the endogenous mRNA. It then cleaves the mRNA about 12 nucleotides from the 3′ terminus of the siRNA (see Sharp et al., Genes Dev., 15:485-490, 2001, and Hammond et al., Nature Rev. Gen., 2:110-119, 2001).

Standard molecular biology techniques can be used to generate siRNAs. Short interfering RNAs can be chemically synthesized, recombinantly produced, e.g., by expressing RNA from a template DNA, such as a plasmid, or obtained from commercial vendors such as Dharmacon. The RNA used to mediate RNAi can include synthetic or modified nucleotides, such as phosphorothioate nucleotides. Methods of transfecting cells with siRNA or with plasmids engineered to make siRNA are routine in the art.

The siRNA molecules used to modulate expression of a glucose transport-related polypeptide can vary in a number of ways. For example, they can include a 3′ hydroxyl group and strands of 21, 22, or 23 consecutive nucleotides. They can be blunt ended or include an overhanging end at either the 3′ end, the 5′ end, or both ends. For example, at least one strand of the RNA molecule can have a 3′ overhang from about 1 to about 6 nucleotides (e.g., 1-5, 1-3, 2-4 or 3-5 nucleotides (whether pyrimidine or purine nucleotides) in length. Where both strands include an overhang, the length of the overhangs may be the same or different for each strand.

The siRNA used to modulate expression of a gene may be identical or substantially identical, e.g., at least 80% (or more, e.g., 85%, 90%, 95%, or 100%) identical, e.g., having 3, 2, 1, or 0 mismatched nucleotide(s), to a target region in the gene's mRNA, and the other strand is complementary to the first strand.

Negative control siRNAs (“scrambled”) generally have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. Controls can also be designed by introducing an appropriate number of base mismatches into the selected siRNA sequence.

To further enhance the stability of the RNA duplexes, the 3′ overhangs can be stabilized against degradation (by, e.g., including purine nucleotides, such as adenosine or guanosine nucleotides or replacing pyrimidine nucleotides by modified analogues (e.g., substitution of uridine 2 nucleotide 3′ overhangs by 2′-deoxythymidine is tolerated and does not affect the efficiency of RNAi). Any siRNA can be used in the methods of modulating a glucose transport-related polypeptide, provided it has sufficient homology to the target of interest. There is no upper limit on the length of the siRNA that can be used (e.g., the siRNA can range from about 21 base pairs of the gene to the full length of the gene or more (e.g., 50-100, 100-250, 250-500, 500-1000, or over 1000 base pairs). In various embodiments, the siRNA has a length of less than 31, 30, 28, 25, or 23 nucleotides.

Isolated Polypeptides

Isolated polypeptides encoded by the glucose transport-related genes described herein are also provided. These polypeptides can be used, e.g., as immunogens to raise antibodies, in screening methods, or in methods of treating subjects, e.g., by administration of the polypeptides. Methods are well known in the art for predicting the translation products of the nucleic acids (e.g., using computer programs that provide the predicted polypeptide sequences and direction as to which of the three reading frames is the open reading frame of the sequence). These polypeptide sequences can then be produced either biologically (e.g., by positioning the nucleic acid sequence that encodes them in-frame in an expression vector transfected into a compatible expression system) or chemically using methods known in the art. The entire polypeptide or a fragment thereof can be used in a method of treatment or to produce an antibody, e.g., that is useful in a screening assay.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as “contaminating protein”). In general, when the protein or biologically active portion thereof is recombinantly produced, it is also substantially free of culture medium, i.e., culture medium represents less than about 20%, 10%, or 5% of the volume of the protein preparation. In general, when the protein is produced by chemical synthesis, it is substantially free of chemical precursors or other chemicals, i.e., it is separated from chemical precursors or other chemicals that are involved in the synthesis of the protein. Accordingly such preparations of the protein have less than about 30%, 20%, 10%, or 5% (by dry weight) of chemical precursors or compounds other than the polypeptide of interest.

Proteins and polypeptides can be assayed to determine the expression level of a gene product of interest. Methods for assaying protein expression are known in the art and include Western blot, immunoprecipitation, and radioimmunoassay.

Biologically active portions of a glucose transport-related polypeptide include polypeptides including amino acid sequences sufficiently identical to or derived from the amino acid sequence of the protein, which include fewer amino acids than the full length protein, and exhibit at least one activity of the corresponding full-length protein. Typically, biologically active portions comprise a domain or motif with at least one activity of the corresponding protein. A biologically active portion of a polypeptide can be, for example, 10, 25, 50, 100, or more amino acids in length. Moreover, biologically active portions, in which other regions of a given protein are deleted, can be prepared by recombinant techniques and evaluated for one or more of the functional activities of the native form of a polypeptide. These short polypeptides can be used in treatments to competitively inhibit activity of the gene products of the genes listed in Table 1 or Table 2 (or to inhibit activity of a gene product that regulates the activity or expression of a gene listed in Table 1 or Table 2).

In some embodiments, glucose transport-related polypeptides have the predicted amino acid sequence encoded by a gene selected from the genes in Tables 1 and 2. Other useful proteins are substantially identical (e.g., at least about 45%, preferably 55%, 65%, 75%, 85%, 95%, or 99%) to the predicted amino acid sequence of a polypeptide encoded by a gene in Tables 1 and 2, and (a) retain the functional activity of the protein of the corresponding naturally-occurring protein yet differ in amino acid sequence due to natural allelic variation or mutagenesis, or (b) exhibit an altered functional activity (e.g., as a dominant negative) where desired.

The comparison of sequences and determination of percent identity between two sequences is accomplished using a mathematical algorithm. The percent identity between two amino acid sequences is determined using the Needleman and Wunsch ((1970) J. Mol. Biol. 48:444-453) algorithm, which has been incorporated into the GAP program in the GCG software package (available on the worldwide web at gcg.com), using either a Blossum 62 matrix or a PAM250 matrix, and a gap weight of 16 and a length weight of 1. The percent identity between two nucleotide sequences is determined using the GAP program in the GCG software package (available on the worldwide web at gcg.com), using a NWSgapdna.CMP matrix and a gap weight of 40 and a length weight of 1.

In general, percent identity between amino acid sequences referred to herein is determined using the BLAST 2.0 program, which is available to the public on the worldwide web at ncbi.nlm.nih.gov/BLAST. Sequence comparison is performed using an ungapped alignment and using the default parameters (Blossum 62 matrix, gap existence cost of 11, per residue gap cost of 1, and a lambda ratio of 0.85). The mathematical algorithm used in BLAST programs is described in Altschul et al., Nucleic Acids Research 25:3389-3402, 1997.

Also provided herein are chimeric or fusion proteins. As used herein, a “chimeric protein” or “fusion protein” comprises all or part (e.g., a biologically active portion) of a glucose transport-related polypeptide operably linked to a heterologous polypeptide (i.e., a polypeptide other than the glucose transport-related polypeptide). In the context of a fusion protein, the term “operably linked” is intended to indicate that the polypeptide and the heterologous polypeptide are fused in-frame to each other. The heterologous polypeptide can be fused to the N-terminus or C-terminus of the glucose transport-related polypeptide.

One useful fusion protein is a GST fusion protein in which the glucose transport-related polypeptide is fused to the C-terminus of GST sequences. Such fusion proteins can facilitate the purification of a recombinant glucose transport-related polypeptide.

In another embodiment, the fusion protein contains a heterologous signal sequence at its N-terminus. For example, the native signal sequence of a glucose transport-related polypeptide can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal is (Sambrook et al., eds., Molecular Cloning: A Laboratory Manual. 2nd ed., Cold Spring Harbor Laboratory, Cold Spring Harbor Laboratory Press, Cold Spring Harbor, N.Y., 1989) and the protein A secretory signal (Pharmacia Biotech; Piscataway, N.J.).

In yet another embodiment, the fusion protein is an immunoglobulin fusion protein in which all or part of a glucose transport-related polypeptide is fused to sequences derived from a member of the immunoglobulin protein family. The immunoglobulin fusion proteins can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a glucose transport-related polypeptide. Inhibition of ligand/receptor interaction may be useful therapeutically, both for treating proliferative and differentiative disorders and for modulating (e.g., promoting or inhibiting) cell survival. Moreover, the immunoglobulin fusion proteins can be used as immunogens to produce antibodies directed against a glucose transport-related polypeptide in a subject, to purify ligands and in screening assays to identify molecules that inhibit the interaction of receptors with ligands.

Chimeric and fusion glucose transport-related polypeptides can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence. Moreover, many expression vectors are commercially available that already encode a fusion moiety (e.g., a GST polypeptide). A nucleic acid encoding a glucose transport-related polypeptide can be cloned into such an expression vector such that the fusion moiety is linked in-frame to the polypeptide.

A signal sequence of a polypeptide can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the described polypeptides having a signal sequence, as well as to the signal sequence itself and to the polypeptide in the absence of the signal sequence (i.e., the cleavage products), are provided herein. In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by methods known in the art. Alternatively, the signal sequence can be linked to the protein of interest using a sequence which facilitates purification, such as with a GST domain.

Also provided are variants of the glucose transport-related polypeptides. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein,

Antibodies

An isolated glucose transport-related polypeptide, or a fragment thereof, can be used as an immunogen to generate antibodies using standard techniques for polyclonal and monoclonal antibody preparation. The full-length polypeptide or protein can be used or, alternatively, antigenic peptide fragments can be used as immunogens. The antigenic peptide of a protein comprises at least 8 (e.g., 10, 15, 20, or 30) amino acid residues of the amino acid sequence of a glucose transport-related polypeptide, e.g., a polypeptide listed in Table 1 or Table 2, and encompasses an epitope of the protein such that an antibody raised against the peptide forms a specific immune complex with the protein.

An immunogen typically is used to prepare antibodies by immunizing a suitable subject, (e.g., rabbit, goat, mouse or other mammal). An appropriate immunogenic preparation can contain, for example, a recombinantly expressed or a chemically synthesized polypeptide. The preparation can further include an adjuvant, such as Freund's complete or incomplete adjuvant, or similar immunostimulatory agent.

Polyclonal antibodies can be prepared as described above by immunizing a suitable subject with a glucose sport-related polypeptide as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein, Nature 256:495-497, 1975, the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985) or trioma techniques. The technology for producing hybridomas is well known (see generally Current Protocols in Immunology, 1994, Coligan et al. (eds.) John Wiley & Sons, Inc., New York, N.Y.). Hybridoma cells producing a monoclonal antibody are detected by screening the hybridoma culture supernatants for antibodies that bind the polypeptide of interest, e.g., using a standard ELISA assay.

As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display libraries can be found in, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 9:1370-1372, 1991; Hay et al., Hum. Antibod. Hybridomas 3:81-85, 1992; Huse et al., Science 246:1275-1281, 1989; Griffiths et al., EMBO J. 12:725-734, 1993.

Additionally, recombinant antibodies, such as chimeric and humanized monoclonal antibodies, comprising both human and non-human portions, which can be made using standard recombinant DNA techniques, are provided herein. Such chimeric and humanized monoclonal antibodies can be produced by recombinant DNA techniques known in the art, for example using methods described in WO 87/02671; European Patent Application 184,187; European Patent Application 171,496; European Patent Application 173,494; WO 86/01533; U.S. Pat. No. 4,816,567; European Patent Application 125,023; Better et al., Science, 240:1041-1043, 1988; Liu et al., Proc. Natl. Acad. Sci. USA, 84:3439-3443, 1987; Liu et al., J. Immunol., 139:3521-3526, 1987; Sun et al., Proc. Natl. Acad. Sci. USA, 84:214-218, 1987; Nishimura et al., Canc. Res., 47:999-1005, 1987; Wood et al., Nature, 314:446-449, 1985; and Shaw et al., J. Natl. Cancer Inst., 80:1553-1559, 1988); Morrison, Science, 229:1202-1207, 1985; Oi et al., Bio/Techniques, 4:214, 1986; U.S. Pat. No. 5,225,539; Jones et al., Nature, 321:552-525, 1986; Verhoeyan et al., Science, 239:1534, 1988; and Beidler et al., J. Immunol., 141:4053-4060, 1988.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, and IgE antibodies. For an overview of this technology for producing human antibodies, see Lonberg and Huszar (Int. Rev. Immunol., 13:65-93, 1995). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al., Biotechnology, 12:899-903, 1994).

An antibody directed against a glucose transport-related polypeptide (e.g., monoclonal antibody) can be used to isolate the polypeptide by standard techniques, such as affinity chromatography or immunuoprecipitation. Moreover, such an antibody can be used to detect the protein (e.g., in a cellular lysate or cell supernatant) to evaluate the abundance and pattern of expression of the polypeptide. The antibodies can also be used diagnostically to monitor protein levels in tissue as part of a clinical testing procedure, e.g., for example, to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Methods of Treatment and Pharmaceutical Compositions

Methods of treating disorders related to glucose metabolism are provided herein. “Treating” includes methods that cure, alleviate, relieve, alter, ameliorate, palliate, improve or affect the disorder, the symptoms of the disorder or the predisposition toward the disorder. The methods can be used in vivo or on cells in culture, e.g., in vitro or ex vivo. For in vivo embodiments, the method is performed in a subject, e.g., a human or animal, and includes administering the agent to the subject under conditions effective to permit the agent to modulate the expression or activity of the polypeptide in a cell.

Agents that modulate expression or activity of a glucose transport-related polypeptide in vitro are further tested in vivo in animal models. For example, a test compound identified as a modulator of a glucose transport-related polypeptide is tested in an animal such as an animal model for obesity or diabetes (e.g., type II diabetes, e.g., ob/ob mice obtained from Jackson Laboratories (Strain Name: B6.V-Lep^{ob}/J), db/db mice; see, e.g., Sima A A F, Shafrir E. Animal Models in Diabetes: A Primer. Taylor and Francis, Publ Amsterdam, Netherlands, 2000). At various time points after administration of the test agent, levels of expression or activity of the glucose transport-related polypeptide and/or levels of glucose, glucose tolerance, and plasma insulin are monitored to determine whether the test compound has a beneficial effect on glucose metabolism, relative to control, i.e., whether the test compound causes a reduction in hyperglycemia or plasma insulin levels.

Data obtained from the cell culture assays and animal studies can be used in formulating an appropriate dosage of any given agent for use in humans. A therapeutically effective amount of an agent will be an amount that delays progression of or improves one or more symptoms of the desired condition, whether evident by improvement in an objective sign (e.g., blood glucose levels) or subjective symptom of the disease. Certain factors may influence the dosage and timing required to effectively treat a subject (e.g., the severity of the disease or disorder, previous treatments, the general health and/or age of the subject and other diseases present).

Compositions useful for modulating expression or activity of the glucose transport-related polypeptides (whether previously known or identified by the screening assays described herein), can be incorporated into pharmaceutical compositions and administered to subjects who have, or who are at risk of developing, a disorder or condition related to glucose metabolism (e.g., related to disregulated glucose metabolism such as type I diabetes, type II diabetes, or obesity). Such compositions will include one or more agents that modulate the expression or activity of the glucose transport-related polypeptide and a pharmaceutically acceptable carrier (e.g., a solvent, dispersion medium, coating, buffer, absorption delaying agent and the like, that are substantially non-toxic). Supplementary active compounds can also be incorporated into the compositions.

Pharmaceutical compositions are formulated to be compatible with their intended route of administration, whether oral or parenteral (e.g., intravenous, intradermal, subcutaneous, transmucosal (e.g., nasal sprays are formulated for inhalation), or transdermal (e.g., topical ointments, salves, gels, patches or creams as generally known in the art). The compositions can include a sterile diluent (e.g., sterile water or saline), a fixed oil, polyethylene glycol, glycerine; propylene glycol or other synthetic solvents; antibacterial or antifungal agents such as benzyl alcohol or methyl parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates; and isotonic agents such as sugars (e.g., dextrose), polyalcohols (e.g., manitol or sorbitol), or salts (e.g., sodium chloride). Liposomal suspensions (including liposomes targeted to affected cells with monoclonal antibodies specific for neuronal antigens) can also be used as pharmaceutically acceptable carriers (see, e.g., U.S. Pat. No. 4,522,811). Preparations of the compositions can be formulated and enclosed in ampules, disposable syringes or multiple dose vials. Where required (as in, for example, injectable formulations), proper fluidity can be maintained by, for example, the use of a coating such as lecithin, or a surfactant. Absorption of the active ingredient can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Where oral administration is intended, the agent can be included in pills, capsules, troches and the like and can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Compositions containing the agents that modulate glucose transport-related polypeptides can be formulated for oral or parenteral administration in dosage unit form (i.e., physically discrete units containing a predetermined quantity of active compound for ease of administration and uniformity of dosage). Toxicity and therapeutic efficacy of compounds can be determined by standard pharmaceutical procedures in cell cultures or experimental animals. One can, for example, determine the LD₅₀ (the dose lethal to 50% of the population) and the ED₅₀ (the dose therapeutically effective in 50% of the population), the therapeutic index being the ratio of LD₅₀:ED₅₀. Agents that exhibit high therapeutic indices are preferred. Where an agent exhibits an undesirable side effect, care should be taken to target that agent to the site of the affected tissue (the aim being to minimize potential damage to unaffected cells and, thereby, reduce side effects). Toxicity and therapeutic efficacy can be determined by other standard pharmaceutical procedures.

As noted above, agents identified and administered according to the methods described here can be small molecules (e.g., peptides, peptidomimetics (e.g., peptoids), amino acid residues (or analogs thereof), polynucleotides (or analogs thereof), nucleotides (or analogs thereof), or organic or inorganic compounds (e.g., heteroorganic or organometallic compounds). Typically, such molecules will have a molecular weight less than about 10,000 grams per mole (e.g., less than about 7,500, 5,000, 2,500, 1,000, or 500 grams per mole). Salts, esters, and other pharmaceutically acceptable forms of any of these compounds can be assayed and, if a desirable activity is detected, administered according to the therapeutic methods described herein. Exemplary doses include milligram or microgram amounts of the small molecule per kilogram of subject or sample weight (e.g., about 1 μg-500 mg/kg; about 100 μg-500 mg/kg; about 100 μg-50 mg/kg; 10 μg-5 mg/kg; 10 μg-0.5 mg/kg; or 1 μg-50 μg/kg). While these doses cover a broad range, one of ordinary skill in the art will understand that therapeutic agents, including small molecules, vary in their potency, and effective amounts can be determined by methods known in the art. Typically, relatively low doses are administered at first, and the attending physician or veterinarian (in the case of therapeutic application) or a researcher (when still working at the clinical development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration.

The invention will be further described in the following examples which do not limit the scope of the invention described in the claims.

### EXAMPLES

### Example 1

### Identification of Polypeptides that Enhance or Inhibit Insulin Action on Glucose Transport Using RNAi-Screening

Cell Culture and Electroporation of 3T3-L1 adipocytes with siRNA Oligonucleotides

3T3-L1 fibroblasts were grown in DMEM supplemented with 10% FBS, 50 μg/ml streptomycin, and 50 units/ml penicillin and differentiated into adipocytes as described (Jiang et al., Proc. Natl. Acad. Sci. USA, 100:7569-7574, 2003; Guilherme et al., J. Biol. Chem., 279:10593-10605, 2004). The 3T3-L1 adipocytes were transfected with siRNA duplexes by electroporation. Briefly, 4 or 5 days after differentiation was initiated, adipocytes were detached from culture dishes with 0.25% trypsin and 0.5 mg of collagenase/ml in PBS and washed twice with PBS. Cells were washed, counted and resuspended at a density of 9×10⁶ cells/ml in PBS. Typically, one 150 mm dish was transfected with 6 different siRNA-duplexes. Resuspended cells (0.15 ml) were placed in 0.2 cm gap cuvette (Bio-Rad®) and mixed with 4 mmoles of each SMARTpool® siRNA-duplexes, purchased from Dharmacon. siRNA oligonucleotides were delivered to the cells by a pulse of electroporation with a Bio-Rad® gene pulser II system at the setting of 0.09 kV and 950 μF capacitance. After electroporation, cells were immediately mixed with 1 ml of fresh complete DMEM media. Cells were then transferred from the cuvette to 3 ml of DMEM media in a 15 ml Falcon™ tube and mixed. Aliquots (125 μl) of this cell suspension were seeded into wells of a 96 well plate. Cells from each electroporation were spread into 12 such wells, placed in an incubator and 2-deoxyglucose uptake was measured 72 hours later. For each 2-deoxyglucose assay, cells were also electroporated with scrambled (6 nmoles), Akt1 and Akt2 (4 and 6 nmoles) and PTEN (6 nmoles) siRNAs, as controls. Each 96 well plate contained 12 wells of each of these 3 controls.

2-Deoxyglucose Uptake Assays

Insulin-stimulated glucose transport in 3T3-L1 adipocytes was estimated by measuring 2-deoxyglucose uptake as described (Guilherme et al., J Biol Chem, 279:10593-10605, 2004). Briefly, siRNA transfected cells were reseeded on 96-well plates and cultured for 72 hours, washed twice and serum-starved for two hours with Krebs-Ringer's Hepes buffer (130 mM NaCl, 5 mM KCl, 1.3 mM CaCl₂, 1.3 mM MgSO₄, 25 mM Hepes, pH 7.4) supplemented with 0.5% BSA and 2 mm sodium pyruvate. Cells were then stimulated with insulin for 30 minutes at 37° C. Glucose uptake was initiated by addition of [1,2-³H] 2-deoxy-D-glucose to a final assay concentration of 500 μM. Cells were incubated for 5 minutes at 37° C. Assays were terminated by three washes with ice-cold Krebs-Ringer's Hepes buffer. Briefly, the plates were dipped into a container with 600 ml of ice-cold Krebs-Ringer's Hepes buffer. Liquids in the wells were drained after each dip by patting the plate on a wad of paper towels. The cells were then solubilized by adding 0.05 ml of 1% SDS per well. Uptake is of [³H] was quantitated using a Microplate scintillation counting instrument. Specific uptake was measured by determining non-specific deoxyglucose uptake in samples incubated in the presence of 20 μM cytochalasin B and subtracting the values obtained for this control from each experimental determination.

Small inhibitory RNAs for approximately 500 different genes were obtained and transfected into adipocytes and tested in deoxyglucose uptake assays described above. A number of genes, when knocked down by transfection of siRNA, resulted in increased or decreased glucose uptake as compared to controls. RNAs targeting the genes listed in Table 1 above strongly increased glucose uptake. RNAs targeting the genes listed in Table 2 strongly decreased glucose uptake.

### Example 2

### Validation of Polypeptides that Enhance or Inhibit Insulin Action on Glucose Transport

To confirm that siRNA-based gene silencing enhanced insulin-stimulated 2-deoxyglucose uptake in adipocytes, further experiments are performed in which the targets identified by screening are independently retested in a Western blot assay that determines the effect of each siRNA on insulin-induced Akt phosphorylation. It has been shown that Akt mediates insulin signaling (see Jiang et al., Proc. Natl. Acad. Sci. USA, 100:7569-7574, 2003; and references cited therein). Phosphorylation of Akt at serine 473 is indicative of activation.

3T3-L1 adipocytes electroporated with siRNA are starved overnight in serum-free DMEM media Cells are then incubated without or with insulin at various concentrations (1, 10, 100 nM) for 30 minutes and harvested with lysis buffer containing 1% SDS. Protein concentrations are quantitated and equivalent amounts of protein from each lysate sample are resolved by SDS-PAGE and transferred to nitrocellulose membranes. Membranes are incubated with antibodies overnight at 4° C. and then with horseradish peroxidase-linked secondary antibodies for 45 minutes at room temperature. Proteins are then detected with an enhanced chemiluminescence kit.

The effect of each siRNA (as compared to scrambled siRNA) on phosphorylation of serine 473 of Akt is analyzed by Western blot. Modulation of GLUT4 expression in the presence of each siRNA is also examined. siRNA that cause an increase in Akt phosphorylation and/or GLUT4 expression indicate that the gene targeted by the siRNA encodes a negative regulator of glucose transport. Inhibition of negative regulators of glucose transport can be beneficial in the treatment of disorders in which insulin signaling is disregulated, e.g., in type II diabetes. Lack of an effect on Akt phosphorylation and/or GLUT4 expression does not necessarily indicate that the gene targeted by the particular siRNA does not regulate glucose transport. Regulators of glucose transport may also effect GLUT4 catalytic activity, translocation of GLUT4 to the plasma membrane, or may effect other glucose transporters such as GLUT1 and GLUT3. These effects can been determined, e.g., by performing assays to determine the amount of glucose transporter protein on the cell surface of fat or muscle cells.

### Example 3

### Evaluating Agents in an Animal Model

Agents that modulate expression or activity of a glucose transport-related polypeptide or nucleic acid encoding the polypeptide in vitro are further tested in vivo in animal models. For example, scrambled siRNA or siRNA that target one or more genes listed in Table 1 are administered to ob/ob mice using hydrodynamic injection as previously described (McCaffrey, Nature, 418:38-39, 2002; see also U.S. Pat. Pub. 20030153519). Ob/ob mice can be obtained from Jackson Laboratories (Strain Name: B6.V-Lep^{ob}/J). At various time points after administration of the siRNA, mRNA levels for the target(s) from Table 1 are measured. Additionally, the siRNA can be labeled and tracked using methods known in the art. Levels of glucose, glucose tolerance, and plasma insulin can also be monitored to determine whether the siRNA has a beneficial effect on glucose metabolism, relative to control, i.e., whether the siRNA causes a reduction in hyperglycemia or plasma insulin levels.

In one embodiment, siRNAs that target cdk7 are designed and generated. Briefly, fragments of a particular length (e.g., 23 nucleotides) within the cdk7 gene sequence are identified, e.g., as described in U.S. Pat. Pub. No. 20040198682. Fragments containing 40-60% GC content and weaker internal fold structure (as determined by in silico analysis) are preferred. Fragments containing strong hairpins and runs of three or more Cs or Gs are avoided. Four or five target fragments are selected and synthesized as siRNA duplexes and screened in vitro to identify the most active siRNAs.

Next, the selected cdk7 siRNA are tested in ob/ob mice in vivo. To perform hydrodynamic injection, each ob/ob mouse is administered 40 micrograms of the selected cdk7 siRNA in 1.8 mL of PBS. The siRNA/PBS solution is injected through the tail vein in 4-5 seconds. Levels of cdk7 expression are determined by examining cdk7 RNA and/or protein levels in tissues 24 hours, 48 hours, 72 hours, or 4 days after injection. Plasma glucose levels in each animal at 1-3 days following treatment are also measured and compared to controls (e.g., glucose levels prior to siRNA treatment and glucose levels in animals treated with PBS and scrambled siRNA). Cdk7 siRNA that reduce hyperglycemia can be useful in treating glucose transport-related disorders such as diabetes and obesity.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A method for identifying a candidate agent that modulates expression or activity of a glucose transport-related polypeptide, the method comprising
(a) providing a sample comprising a glucose transport-related polypeptide or a nucleic acid encoding the polypeptide, wherein the glucose transport-related polypeptide is a gene product of a gene in Table 1 or Table 2;
(b) contacting the sample with a test compound;
(c) evaluating expression or activity of the glucose transport-related polypeptide in the sample; and
(d) comparing the expression or activity of the glucose transport-related polypeptide of (c) to expression or activity of the glucose transport-related polypeptide in a control sample lacking the test compound, wherein a change in glucose transport-related polypeptide expression or activity indicates that the test compound is a candidate agent that can modulate the expression or activity of the glucose transport-related polypeptide.

2. The method of claim 1, wherein the glucose transport-related polypeptide is a gene product of a gene in Table 1.

3. The method of claim 1, wherein the glucose transport-related polypeptide is a gene product of a gene in Table 2.

4. The method of claim 1, wherein the sample is a cell.

5. The method of claim 4, wherein the cell is an adipocyte.

6. The method of claim 1, wherein the sample is a cell-free sample.

7. The method of claim 1, wherein evaluating comprises performing a cell-free assay.

8. The method of claim 1, wherein evaluating comprises determining whether glucose transport is modulated in the presence of the test compound.

9. The method of claim 1, wherein the glucose transport-related polypeptide is a human glucose transport-related polypeptide.

10. The method of claim 1, wherein evaluating comprises determining glucose uptake.

11. The method of claim 1, wherein the test compound is selected from the group consisting of a polynucleotide, a polypeptide, a small non-nucleic acid organic molecule, a small inorganic molecule, and an antibody.

12. The method of claim 1, wherein the test compound is selected from the group consisting of an antisense oligonucleotide, a small interfering RNA (siRNA), a DNA encoding an siRNA, and a ribozyme.

13. The method of claim 8, wherein modulation of glucose transport is evaluated using an antibody.

14. The method of claim 8, wherein glucose transport is increased in the presence of the test compound.

15. The method of claim 8, wherein glucose transport is decreased in the presence of the test compound.

16. The method of claim 1, wherein the glucose transport-related polypeptide is a kinase.

17. The method of claim 16, wherein the evaluating comprises determining phosphorylation of a substrate by the kinase.

18. A method for modulating glucose transport in a cell, the method comprising:
providing a cell;
contacting the cell with an agent that modulates expression or activity of a gene product in Table 1 or Table 2, thereby modulating glucose transport in the cell.

19. The method of claim 18, wherein the agent decreases expression or activity of a gene product of a gene in Table 1.

20. The method of claim 18, wherein the agent increases expression or activity of a gene product of a gene in Table 2.

21. The method of claim 18, wherein the agent is selected from the group consisting of a polynucleotide, a polypeptide, a small non-nucleic acid organic molecule, a small inorganic molecule, and an antibody.

22. The method of claim 21, wherein the agent is a small inhibitory RNA.

23. The method of claim 21, wherein the agent is selected from the group consisting of an antisense oligonucleotide, an siRNA, a DNA encoding an siRNA, and a ribozyme.

24. The method of claim 18, further comprising contacting the cell with a second agent that modulates expression or activity of a glucose transport-related polypeptide.

25. The method of claim 18, wherein the cell is contacted in vitro.

26. The method of claim 18, wherein the cell is contacted in vivo.

27. A method for increasing insulin-stimulated glucose uptake in a subject, the method comprising:
administering to the subject an agent that decreases expression or activity of a gene product of a gene in Table 1 in an amount sufficient to modulate glucose metabolism in a cell of the subject hereby increasing insulin-stimulated glucose uptake in the subject.

28. The method of claim 27, wherein the subject is at risk for or suffering from a disorder or condition related to glucose metabolism.

29. The method of claim 28, wherein the disorder or condition is type I diabetes, type II diabetes, or obesity.

30. A method for modulating glucose metabolism in a subject, the method comprising:
administering to the subject an agent that increases expression or activity of a gene product of a gene in Table 2 in all amount sufficient to modulate glucose metabolism in a cell of the subject, thereby modulating glucose metabolism in the subject.

31. The method of claim 30, wherein the subject is at risk for or suffering from a disorder or condition related to glucose metabolism.

32. The method of claim 31, wherein the disorder or condition is type I diabetes, type II diabetes, or obesity.

33. A composition comprising an siRNA or a nucleic acid encoding an siRNA that targets an RNA encoded by a gene of Table 1.

34. A composition comprising an antisense nucleic acid that inhibits the function of a gene product of a gene of Table 1.
